(19) United States
(12) Patent Application Publication
(10) Pub. No.: US 2006/0110475 A1
(43) Pub. Date: May 25, 2006

(54) Composition containing in combination at least one bourd oil and at least one borage oil, use thereof as medicine, as dermatological or dermato-cosmetic agent

(76) Inventor: Marc Schwaller, Les Escaldes (AD)

Correspondence Address: BIRCH STEWART KOLASCH & BIRCH, PO BOX 747, FALLS CHURCH, VA, 22040-0747, US

(21) Appl. No.: 10/527,810

(22) PCT Filed: Sep. 11, 2003
(86) PCT No.: PCT/FR03/02693

§ 371 Date: Jan. 17, 2006

(30) Foreign Application Priority Data
Sep. 12, 2002 (FR) 02/11351

(51) Int. Cl. A61K 36/48; A61K 36/28; A61K 36/30

(52) U.S. Cl. 424/757; 424/776; 424/764

(57) ABSTRACT

The present invention relates to a composition comprising, in combination: at least one marrow oil and at least one borage oil. The invention also relates to such a composition, for its use as a medicinal product, as a dermatological or dermocosmetic agent, or as a nutraceutical agent, especially for its use in preventing or treating alopecia, acne, hirsutism, seborrhoea or body odour.

The present invention relates to a novel composition comprising, in combination, at least one marrow oil, at least one borage oil and, optionally, at least one compound chosen from a soybean extract and a nettle extract. A subject of the invention is also such a composition for its use as a medicinal product, as a dermatological or dermocosmetic agent, or as a nutraceutical agent (food supplement), especially for its use in the prevention or treatment of alopecia, acne, hirsutism, seborrhoea or body odour.

Marrow, whose Latin name is Cucurbita pepo, belongs to the Cucurbitacea family. Marrow is a large annual herbaceous plant with a very branchy angular stem. The leaves are simple, alternate and extensively petiolated. The yellow flowers are unisexual. The fruit is a large voluminous or oblong berry with a fleshy, spongy pulp and is of variable colour. The seeds are numerous, flat and ovoid. The pips are chemically composed on average of 35 to 50% oil, 25 to 40% protein, 10% pectins and 4 to 5% minerals. The oil, which is rich in tocopherols, carotenoids, unsaturated fatty acids (70 to 80% of the total fatty acids) and phytosterols, is the most beneficial part, and is commonly used, especially for its nutritional value.

Alongside this food use, marrow oil is conventionally used orally as a supplement for treating prostate functional disorders and diuresis difficulties. It is thus stated that marrow oil acts as a prostate decongestant. Among the prostate functional disorders that may especially be mentioned are benign hypertrophy of the prostate and prostate adenoma. Marrow oil is moreover used as an antiparasitic agent or as an anthelminthic agent.

The Applicant has discovered, surprisingly, that the combination of an oil extracted from marrow with an oil extracted from borage, in compositions such as pharmaceutical, dermatological, cosmetic or food compositions, allows various skin disorders and complaints to be treated, and allows this to be achieved much more efficiently than with the compositions used hitherto, in particular compositions containing marrow oil as sole active agent.

Borage, whose Latin name is Borago officinalis, belongs to the Boraginacea family. Borage is an annual herbaceous plant with large basal leaves and beautiful blue flowers. The oil from borage seeds is rich in polyunsaturated fatty acids, especially gamma-linolenic acid, and has already been used to improve skin moisturization and to treat premenstrual pain, rheumatism and eczema. However, borage oil has never been used in combination with marrow oil in compositions for the skin or the scalp.

Now, the Applicant has shown that the following active agents: marrow oil and borage oil, to which may be added at least one other compound chosen from a nettle extract, a soybean extract, or mixtures thereof, can be used in combination in compositions such as pharmaceutical, dermatological, cosmetic, nutraceutical or food compositions, especially in the prevention or treatment of alopecia, acne, hirsutism, seborrhoea or body odour.

The marrow oil and borage oil according to the present invention, and also the extracts of nettle and of soybean, are natural products, and are found to be cosmetically, pharmaceutically, dermatologically and nutraceutically acceptable compounds, which are neither aggressive nor toxic nor irritant to the skin and which are hypoallergenic, calmative, moisturizing and antiinflammatory to the skin. Furthermore, these compounds are obtained by standard extraction processes, and are commercially available.

One subject of the present invention is thus a composition comprising, in combination:

(a) at least one marrow oil, and

(b) at least one borage oil.

Advantageously, according to the present invention, the composition also comprises at least one compound chosen from a nettle extract (c) and a soybean extract (d). The composition according to the present invention may thus contain as active agents a marrow oil (a) and a borage oil (b), or alternatively a marrow oil (a) and a borage oil (b) in combination with either a nettle extract (c) or a soybean extract (d), or a nettle extract (c) and a soybean extract (d).

In one particular embodiment of the present invention, the marrow oil (a) is an oil extracted from marrow pips and/or the borage oil (b) is an oil extracted from borage seeds. The processes for extracting these two types of oil are conventional.

The marrow pip oil and the borage oil may thus be obtained via different processes, used separately or in combination:

via simple cold-pressing of the pips or seeds, allowing all the lipophilic components present in the pips to be collected;

via organic solvents such as ethanol, methanol, dichloromethane, chloroform and hexane, which may result in a certain level of selectivity in the extraction;

via carbon dioxide in supercritical form.

The composition according to the invention may use an oil obtained by one or other of these processes, with a preference for cold-pressing, which is the only method that guarantees the obtention of all the lipophilic components of the seed, and that also guarantees the absence of any degradation of certain components as a result of the heating required to evaporate off the solvents.

In another particular embodiment of the present invention, the nettle extract (c) is an extract of nettle roots, advantageously in powder form. This extract is conventionally obtained by macerating nettles in a solvent, which may be water, a solvent such as ethanol or hexane, or, advantageously, an aqueous-alcoholic mixture, followed by conversion into powder by atomization.

In another particular embodiment of the present invention, the soybean extract (d) is soybean lecithin, advantageously obtained from soybean seed oil, or an isoflavone-rich extract, advantageously in powder form. The isoflavone-rich extract is advantageously an aqueous-alcoholic soybean extract. The expression “isoflavone-rich extract” means, for the purposes of the present invention, an extract containing at least 2% by weight and preferably from 10% to 40% by weight of isoflavones relative to the total weight of the extract.

According to one particular characteristic of the present invention, the marrow oil (a) is present in a concentration of between 20% and 40% and advantageously between 25% and 35% by weight relative to the total weight of the composition.

According to another particular characteristic of the present invention, the borage oil (b) is present in a concentration of between 10% and 30% and advantageously between 15% and 30% by weight relative to the total weight of the composition.

According to another particular characteristic of the present invention, the nettle extract (c) is present in a concentration of between 0 and 60%, advantageously between 25% and 60% and even more advantageously between 35% and 55% by weight relative to the total weight of the composition.

According to another particular characteristic of the present invention, the soybean extract (d) is present in a concentration of between 0 and 10% and advantageously between 0 and 5% by weight relative to the total weight of the composition.

In one particular embodiment of the present invention, the composition is formulated for oral administration. The composition may thus be in the form of a wafer capsule, a gel capsule, a tablet, a granule, a chewing paste, a gum, an oil or an oily jelly. When the composition is in the form of a soft capsule or a gel capsule, the casing of these soft capsules or of these gel capsules may consist of animal gelatin or of a material of plant origin (cellulose or starch derivative, or plant protein). When the composition is in the form of a gel capsule, a tablet or a granule, the mixture of active agents may be bound to a pulverulent support such as silica, cellulose or maltodextrin.

Advantageously, according to the present invention, the composition is an oral composition containing the mixture: marrow pip oil (a), borage oil (b), nettle root extract (c) and soybean lecithin (d), and, if necessary, technological additives such as thickeners or antioxidants, and is advantageously in the form of a soft capsule or a gel capsule.

The composition may also be a food composition or a food supplement, such as a cereal bar, a powder for dilution in water, of the type such as instant coffee, instant tea or instant chocolate, a cream dessert, and margarine. In this case, the mixture of active agents may be bound to a pulverulent support.

In another particular embodiment of the present invention, the composition is formulated for topical or rectal administration. In this case, and when the nettle extract (c) is in powder form, the fraction of nettle powder not dissolved in the oil mixture is advantageously removed. The composition according to the present invention may thus be applied to the skin or to the scalp, and may be in the form of a cream, an ointment or an oil. The composition may also be a rectal composition, and may especially be in the form of suppositories or cannulas.

Advantageously, according to the present invention, when the nettle extract is in powder form, the composition may be obtained according to the process comprising the step of macerating the nettle powder (c) in a mixture of marrow oil (a) and borage oil (b), to which soybean lecithin is optionally added. Irrespective of the mixture used for the maceration (mixture of oils or mixture of oils and of soybean lecithin), if an isoflavone-rich soybean extract is used, it will be added after maceration.

Advantageously, according to the present invention, the composition is titrated so as to allow the administration of a daily dose of 10 mg to 5 g, and preferably about 400 mg, of marrow oil (a) per day, from 10 mg to 5 g, and preferably about 300 mg, of borage oil (b) per day, from 10 mg to 5 g, and preferably about 400 mg, of nettle extract (c) per day, and from 5 mg to 10 g of soybean extract (d) per day, preferably about 40 mg when the soybean extract (d) is soybean lecithin.

Advantageously, according to the present invention, the composition is a pharmaceutical, dermatological, cosmetic, nutraceutical or food composition, and may comprise any pharmaceutically, dermatologically, cosmetically or nutraceutically acceptable suitable vehicle or excipient, and also conventional additives, known to those skilled in the art.

The composition according to the present invention may contain other active agents, such as an oil of Serenoa repens having inhibitory activity on 5-α reductase; an antioxidant, catechol-rich extract of green tea for combating apoptosis of the hair follicles; a zinc salt, such as zinc acetate, chloride, citrate, gluconate, lactate, oxide, carbonate or sulphate, or a chelated form of zinc such as zinc-amino acid chelate, which also has inhibitory activity on 5-α reductase; group B vitamins, such as vitamin B1 or thiamine (especially in hydrochloride or mononitrate form), vitamin B2 or riboflavin (especially in pure form or in sodium phosphate form), vitamin B3 or niacin (especially in nicotinic acid or nicotinamide form), vitamin B5 or pantothenic acid (especially in the form of calcium or sodium pantothenate or in the form of dexpantothenol), vitamin B6 or pyridoxine (especially in hydrochloride or phosphate form), vitamin B12 or cobalamin (especially in the form of cyanocobalamin and hydroxycobalamin), vitamin H or biotin; and sulphur-containing amino acids, which may play an important role as a nutrient for the hair bulb (methionine and cystine).

A subject of the present invention is also the compositions described above for their use as medicinal products, as dermatological or dermocosmetic agents, or as nutraceutical agents (food supplements).

A subject of the present invention is also the compositions described above for their use in the prevention or treatment of alopecia, acne, hirsutism, seborrhoea or body odour.

Hair loss may be caused by various factors and may give rise to a simple aesthetic displeasure or may constitute a veritable pathology. The composition according to the present invention allows various existing forms of alopecia to be treated.

Before listing the various existing forms of alopecia, it is recalled that the hair bulb is in permanent evolution between three phases: the anagenic phase (growth phase), the telogenic phase (resting phase) and the catagenic phase (degeneration phase). In the normal state, more than 90% of hairs are in the anagenic stage. “Hair loss” is the result of two physiological mechanisms that are often correlated: firstly, an abnormal increase in the percentage (20 to 40% instead of 10%) of hair in the telogenic phase, the telogenic “hairs” then being limited to a “down”, and, secondly, the degeneration of the bulb (catagenic phase), leading to the death and loss of the hair.

These physiological mechanisms appear to have several origins:

hormonal: the hair bulb is very sensitive to androgens and especially to testosterone derivatives. An excess of activity of 5-a reductase, the enzyme that converts testosterone into dihydrotestosterone, which is hormonally the more active form, appears to promote the passage of the hair into the telogenic phase. Androgenetic alopecia is caused by an abnormally high enzymatic activity of 5-a reductase in the scalp. It is on the basis of this finding that the most recent medicinal product for combating alopecia was developed: Finasteride, which is a 5-a reductase inhibitor.

circulatory: a reduction in the capillary circulation in the hair bulb results in a slowing-down of its growth, or even its degeneration. This is the recognized mechanism of action of a medicinal product widely used in alopecia: Minoxidil, which is a peripheral vasodilator.

immunological: rapid and substantial hair loss may be caused by an autoimmune reaction directed against the hair bulb: this is diffuse alopecia or pelada (alopecia aerata) which may remain localized or be generalized to the entire scalp (alopecia totalis), or even to all the hairs of the body (alopecia universalis). Its current medicinal treatment consists essentially of antiinflammatories (corticoids) and immunosuppressants (cyclosporin).

As the present invention is a combination of several natural substances of plant origin, it is probable that its clinical efficacy is the result of several mechanisms of action, the main ones being:

regulation of the excess 5-a reductase: specifically, it has been demonstrated that several components of the formulation have inhibitory activity on 5-a reductase (marrow oil, nettle root and borage oil). This activity is partly associated with the phytosterols contained in these three plant extracts, but also with the unsaturated fatty acids contained in the two oils; this action might result from a change in the membrane environment of the enzyme.

antiinflammatory activity: mainly via the unsaturated fatty acids of the two oils.

This twofold mechanism of action makes it possible to explain the activity observed in the treatment of different forms of alopecia: androgenetic alopecia and diffuse alopecia (pelada), but also in other skin pathologies of both hormonal and inflammatory origin: acne, hirsutism, seborrhoea and body odour.

The examples that follow are intended to illustrate the invention without in any way limiting its scope. Unless otherwise specified, the percentages indicated in the examples that follow are percentages by weight.

### EXAMPLES OF COMPOSITIONS ACCORDING TO THE PRESENT INVENTION AND OF PROCESSES FOR PREPARING THEM

### Example 1

### Composition, in Capsule Form, Containing a Macerate of Nettle Root (c) Powder in a Mixture of Marrow Pip Oil (a), Borage Seed Oil (b) and Soybean Lecithin (d)—Preparation Process

a) Dissolution of the Lipophilic Components of the Nettle Root Powder in the Mixture of Marrow Pip Oil, Borage Oil and Lecithin:

Marrow pip oil: 100 kg

Nettle root powder: 200 kg

Borage oil: 75 kg

Lecithin: 10 kg

In a heating vessel placed under partial vacuum:

mix the marrow pip oil, the borage oil, the lecithin and, optionally, an antioxidant (for example tocopheryl acetate) and then bring the temperature to 70° C.,

introduce the finely ground (particle size of less than 300 μm) nettle root powder with stirring, and keep stirring for 2 hours,

cool to room temperature with stirring, and then, optionally,

package the mixture obtained in a leaktight vat under a nitrogen atmosphere.

b) Manufacture of the Soft Capsules

The process for manufacturing the soft capsules is entirely standardized: the mixture described above may be injected directly at the time of hot-sealing of the two gelatin shells of the soft capsule in suitable rotary moulds. The sealed casing of the soft capsules is then dried in a stream of hot air, a small amount of lubricant generally being added to prevent the soft capsules from adhering together.

By way of example, each soft capsule may contain:

Marrow pip oil: 200 mg

Nettle root powder: 400 mg

Borage oil: 150 mg

Lecithin: 20 mg

### Example 2

### Composition, in Capsule Form, Containing a Mixture of Marrow Pip Oil (a), Borage Seed Oil (b), Nettle Root Extract (c) and Soybean Lecithin (d) Process for Preparing the Composition by Simple Mixing of the Components

a) Mixing of the Components:

Marrow pip oil: 100 kg

Nettle root extract: 100 kg

Borage oil: 75 kg

Lecithin: 10 kg

Into a tank equipped with a stirring system:

introduce successively the marrow pip oil, the borage oil and the lecithin; moderate heating (40 to 50° C.) and/or preheating of the lecithin may be necessary in order to homogenize it fully,

while maintaining the stirring, gradually incorporate the nettle root extract and then, optionally,

package the mixture obtained in a leaktight vat under a nitrogen atmosphere.

b) Manufacture of the Soft Capsules

The process for manufacturing the soft capsules is identical to that of Example 1, the composition per capsule being:

Marrow pip oil: 200 mg

Nettle root extract: 200 mg

Borage oil: 150 mg

Lecithin: 20 mg

Evaluation of the Activity Induced by Administering Compositions According to the Present Invention to Combat Alopecia in Man

### 1. EXPERIMENTAL PROTOCOL

A clinical study was performed on 70 male volunteers with alopecia of androgenetic type, who followed for 6 months a treatment consisting of taking, morning and evening, a capsule containing one of the compositions below:
Group 1: Marrow pip oil: 200 mg.
Group 2: Nettle root extract: 200 mg.
Group 3: Borage oil: 150 mg.
Group 4: Soybean lecithin: 20 mg.
Group 5: * Marrow pip oil: 200 mg, and
   * Borage oil: 150 mg.
Group 6: * Marrow pip oil: 200 mg,
   * Nettle root extract: 200 mg, and
   * Borage oil: 150 mg.
Group 7: * Marrow pip oil: 200 mg,
   * Nettle root extract: 200 mg,
   * Borage oil: 150 mg, and
   * Soybean lecithin: 20 mg.
   Simple mixture of the 4 components.
Group 8: * Marrow pip oil: 200 mg,
   * Borage oil: 150 mg,
   * Soybean lecithin: 20 mg, and
   * Nettle root powder: 400 mg.
   Maceration at 70° C. for 2 hours of the nettle root powder in the mixture: marrow pip oil+borage oil+lecithin.

### 2. TESTS PERFORMED

For each volunteer, a trichogram was performed on the same region of 1 cm² of scalp before participation in the study, and after 3 months of treatment: this made it possible to determine the total number of hairs and the number of hairs in the anagenic phase (anagenic hairs/cm²).

Using these experimental values, it was possible to calculate the number of hairs in the telogenic phase (telogenic hairs/cm²=number of hairs/cm²−anagenic hairs/cm²) and to deduce therefrom the anagenic/telogenic ratio (number of hairs in the anagenic phase/number of hairs in the telogenic phase).

### 3. RESULTS

The results of the number of hairs in the telogenic phase and of the anagenic/telogenic ratio are given in Table 1 below:

TABLE 1

| | Total number of hairs/cm² | | | Anagenic/telogenic ratio | | |
|---|---|---|---|---|---|---|
| | Before | After | | Before | After | |
| | treat- | treat- | | treat- | treat- | |
| | ment | ment | Change | ment | ment | Change |
| 1 | 167.2 ± | 171.2 ± | 2.4% | 1.96 ± | 2.16 ± | +10.2% |
| | 3.0 | 4.9 | NS | 0.14 | 0.22 | NS |
| 2 | 162.8 ± | 166.6 ± | +2.3% | 2.02 ± | 2.30 ± | +13.9% |
| | 7.2 | 9.6 | NS | 0.21 | 0.28 | NS |
| 3 | 168.4 ± | 171.6 ± | +1.9% | 2.18 ± | 2.26 ± | +3.7% |
| | 4.3 | 4.5 | NS | 0.17 | 0.22 | NS |
| 4 | 167.0 ± | 168.6 ± | +0.9% | 2.02 ± | 2.12 ± | +4.9% |
| | 5.4 | 3.7 | NS | 0.15 | 0.14 | NS |
| 5 | 166.6 ± | 172.2 ± | +3.4% | 2.04 ± | 2.46 ± | +20.5% |
| | 9.4 | 8.7 | S(p < 0.05) | 0.41 | 0.34 | S(p < 0.05) |
| 6 | 164.8 ± | 169.8 ± | +3.0% | 2.16 ± | 2.60 ± | +20.3% |
| | 6.8 | 9.1 | S(p < 0.05) | 0.22 | 0.14 | S(p < 0.05) |
| 7 | 163.2 ± | 169.6 ± | +3.9% | 1.92 ± | 2.36 ± | +22.9% |
| | 9.3 | 8.3 | S(p < 0.05) | 0.18 | 0.28 | S(p < 0.01) |
| 8 | 167.0 ± | 175.0 ± | +4.8% | 1.90 ± | 2.48 ± | +30.5% |
| | 7.5 | 3.2 | S(p < 0.05) | 0.20 | 0.20 | S(p < 0.01) |

It is observed that the number of hairs is very slightly increased after treatment with the marrow pip oil, the borage oil and the nettle root extract alone (groups 1, 2 and 3). This increase becomes statistically call significant with group 5, which combines the marrow pip oil and the borage oil.

The anagenic/telogenic ratio increases after treatment with the marrow pip oil, the borage oil and the nettle root extract alone (groups 1, 2 and 3) but insignificantly. The increase is substantial and significant for groups 5 to 8 after treatment.

It is interesting to note that the largest increase in this ratio is obtained for group 8, using a maceration of nettle root powder in the mixture of marrow pip oil, borage oil and soybean lecithin.

Finally, it was observed after the treatment that a remnant of the effect was manifested, a resumption of hair loss not appearing, on average, until 1 to 3 months after stopping the treatment. This very advantageous aspect differentiates the present invention from most of the treatments available to date.

### 4. CONCLUSIONS

The clinical tests (total number of hairs/cm² and anagenic/telogenic ratio) thus demonstrate a very moderate activity of marrow pip oil alone (400 mg/day), of nettle root extract alone (400 mg/day), of borage oil alone (300 mg/day) or of soybean lecithin alone (40 mg/day).

On the other hand, entirely unexpectedly, by combining marrow oil with borage oil, to which may be added a nettle root extract and/or a soybean extract, genuine anti-alopecia activity could be observed.

Even more surprisingly, by macerating a nettle root powder in the mixture: marrow pip oil—borage oil—lecithin, the product obtained developed even higher clinical activity at the same dosage. After checking the absence of activity of lecithin at the dose tested, the observation could not be avoided that this novel manufacturing process, comprising the step of macerating nettle powder in the mixture of oil or in the mixture of oils and of lecithin, gave this preparation unexpected properties.

The fact that the anti-alopecia activity is manifested to an even greater extent when the components are used according to the above process may have several explanations, and may especially be associated with:

an incapacity of the human body to assimilate certain hydrophobic components present in marrow pip oil, or in nettle root powder, administered alone, whereas the solubilizing and complexing power (formation of micelles) of lecithin is thought to allow better assimilation (bioavailability) of these components and thus their beneficial action on the follicles, and/or

a selective extraction of certain components present in nettle root by the marrow pip oil and borage oil emulsified by the lecithin, and/or

a synergism of activity of each of the necessary components to observe a significant clinical improvement, and/or

the capacity of the components contained in the formulation to exert antiinflammatory activity and/or to modulate immunity.

1. Composition comprising, in combination:
(a) at least one marrow oil, and
(b) at least one borage oil.

2. Composition according to claim 1, characterized in that it also comprises at least one compound chosen from a nettle extract (c) and a soybean extract (d).

3. Composition according to claim 1 or 2, characterized in that the marrow oil (a) is an oil extracted from marrow pips and/or the borage oil (b) is an oil extracted from borage seeds.

4. Composition according to claim 1, characterized in that the nettle extract (c) is an extract of nettle roots, advantageously in powder form.

5. Composition according to claim 1, characterized in that the soybean extract (d) is soybean lecithin or an isoflavone-rich extract, advantageously in powder form.

6. Composition according to claim 1, characterized in that the marrow oil (a) is present in a concentration of between 20% and 40% and advantageously between 25% and 35% by weight relative to the total weight of the composition.

7. Composition according to claim 1, characterized in that the borage oil (b) is present in a concentration of between 10% and 30% and advantageously between 15% and 30% by weight relative to the total weight of the composition.

8. Composition according to claim 1, characterized in that the nettle extract (c) is present in a concentration of between 0 and 60% and advantageously between 35% and 55% by weight relative to the total weight of the composition.

9. Composition according to claim 1, characterized in that the soybean extract (d) is present in a concentration of between 0 and 10% and advantageously between 0 and 5% by weight relative to the total weight of the composition.

10. Composition according to claim 1, characterized in that the composition is formulated for oral administration.

11. Composition according to claim 10, characterized in that the composition is in the form of a wafer capsule, a gel capsule, a tablet, a granule, a chewing paste, a gum, an oil or an oily jelly.

12. Composition according to claim 10, characterized in that the composition is a food composition or a food supplement.

13. Composition according to claim 1, characterized in that the composition is formulated for topical or rectal administration.

14. Composition according to claim 4, characterized in that it can be obtained according to the process comprising the maceration of the nettle powder (c) in a mixture of marrow oil (a) and borage oil (b), to which soybean lecithin is optionally added.

15. A medicinal product, a dermatological agent, a dermocosmetic agent, or a nutraceutical agent comprising the composition according to claim 1.

16. a method for preventing or treating alopecia, acne, hirsutism, seborrhoea, or body odor, comprising administering the composition according to claim 1.
